(19) United States
(12) Patent Application Publication
(10) Pub. No.: US 2022/0379010 A1
(43) Pub. Date: Dec. 1, 2022

(54) FLUID DELIVERY SYSTEMS AND METHODS OF TREATMENT

(71) Applicant: ALCYONE THERAPEUTICS, INC., Lowell, MA (US)

(72) Inventors: Bryn Martin, Moscow, ID (US); Mohammadreza Khani, Moscow, ID (US); Lucas Sass, Moscow, ID (US); Ostin Arters, Moscow, ID (US); Stuart Sater, Troy, ID (US); Deep Arjun Singh, Cambridge, MA (US); PJ Anand, Lowell, MA (US); Thomas T. Washburn, Lancaster, MA (US); Krishna Subramanian, Mechanicsville, VA (US)

(21) Appl. No.: 17/748,199

(22) Filed: May 19, 2022

Related U.S. Application Data
(60) Provisional application No. 63/201,941, filed on May 19, 2021.

(51) Int. Cl. A61M 5/14; A61M 5/168; A61M 5/00; A61M 5/172; C12N 15/113

(52) U.S. Cl. CPC A61M 5/1409; A61M 5/16877; A61M 5/007; A61M 5/1723; C12N 15/113; A61M 2210/0693; A61M 2005/1726; A61M 2205/3344; A61M 2210/1003; A61M 2005/1403; C12N 2310/141; C12N 2320/32

(57) ABSTRACT

The disclosed systems and methods are configurable central nervous system (CNS) delivery solutions for therapeutics, such as genetic medicines. The systems and methods first infuse a therapeutic bolus within intrathecal space and subsequently infuse a flush fluid to move the therapeutic bolus rostrally toward a target area and achieve a desired spread in the spine and/or brain. The second location can be at a location caudal to the delivery location of the therapeutic bolus.

### CROSS-REFERENCE TO RELATED APPLICATION

This application claims the priority benefit of U.S. Provisional Application No. 63/201,941, filed May 19, 2021, which is hereby incorporated by reference in its entirety.

### FIELD OF THE DISCLOSURE

The present disclosure generally relates to fluid delivery systems and methods of treatment and, more particularly, to central nervous system fluid delivery systems and methods of treatment.

### BACKGROUND

Many systems of the human body depend upon careful regulation of fluid pressure, volume, flow, and metabolite balance. For example, the intrathecal space is the fluid-filled compartment located between the pia mater surrounding the spinal cord and the arachnoid mater adjacent to the dura. The intrathecal space contains cerebrospinal fluid, a clear water-like fluid with viscosity typically close to water at body temperature. Alterations in intrathecal pressure can result in, e.g., reduced local spinal cord tissue blood flow, reduced metabolite delivery to the spinal cord, and changes in intracranial pressure. Delivery or removal of fluids to the intrathecal space is challenging, as a needle (optionally associated with a catheter) is manually inserted into the spine of a patient. Other dangers associated with intrathecal drug administration include a) infection associated with aseptic instruments, b) inflammatory response of the neural tissue to foreign materials, c) loss of fluid from the needle leading to decreased intrathecal and intracranial pressure and hypotensive headaches, d) incorrect positioning of needle outside of intrathecal space in epidural space, e) injection of fluid/drug too quickly leading to increased intrathecal and intracranial pressure, f) inflammatory response of neural tissue due to injection of foreign fluid, g) neural tissue damage from needle, h) prolonged leakage of cerebrospinal fluid from the needle hole, and many other issues. In combination, these risks are significant with respect to intrathecal injections. The training and time required to manually adapt existing drug delivery devices to different anatomical sites and specific treatments represents a significant burden to clinicians.

Nevertheless, intrathecal administration is a valuable tool for introducing therapeutic agents into the cerebral spinal fluid (CSF), which allows an ability for widespread or concentrated biodistribution throughout the central nervous system. Indeed, therapeutics administered to CSF are distributed to the brain and spinal cord, thereby bypassing the blood-brain barrier that strongly limits the range of drugs that can be delivered to the CNS tissue.

### SUMMARY

In accordance with a first example, a method for fluid delivery to a target area in a central nervous system of a patient is described that includes infusing a therapeutic bolus into an intrathecal space of a patient in a first location and subsequently infusing a flush fluid into the intrathecal space of the patient in a second location at one or more flush flow rates, where the one or more flush flow rates are based on at least one of patient anatomy or physiology data.

In the above example, the one or more flush flow rates can be calculated using: estimated or measured steady-streaming fluid velocities of cerebrospinal fluid within the intrathecal space of the patient between the first location and the target area divided into one or more axial sections; and estimated or measured axial cross-sectional areas of the one or more axial sections. Optionally, the one or more flush flow rates can be based on averages of the steady-streaming fluid velocities or maximum values of the steady-streaming fluid velocities in the one or more axial sections. Optionally, the one or more flush flow rates can be calculated using a predetermined percentage of the averages of the streaming fluid velocities or the maximum values of the steady-streaming fluid velocities in the one or more axial sections.

In the above examples, the at least one of patient anatomy or physiology data can include one or more of: patient age, patient sex, patient size, patient CSF volume, patient CSF dynamics, patient respiration data, patient sleep data, patient anatomical geography, heart rate, or disease. Optionally, the one or more steady-streaming fluid velocities can be estimated by a central nervous system computational or in vitro model for the patient using the at least one of patient anatomy or physiology data as input.

In the above examples, a volume of the flush fluid can correspond to a volume of the cerebrospinal fluid between the first location and the target area. Optionally, the method can include imaging the patient to determine the volume of the cerebrospinal fluid between the first location and the target area.

In the above examples, the method can include one or more of the following options: the at least one of patient anatomy or physiology data can include data obtained from patient imaging and tests, and computations performed on the patient imaging and tests; the target area can be the brain, the spine or combinations thereof; the method can include measuring CSF pressure of the patient with a pressure sensor and stopping the infusion of the flush fluid or reducing the one or more flush flow rates in response to determining that the CSF pressure exceeds a predetermined threshold or drops below a predetermined threshold; the method can include measuring CSF pressure of the patient with a pressure sensor and beginning the infusion of the flush fluid in response to determining that the CSF pressure indicates an ascending or descending phase of a waveform; the second location can be spaced caudally from the first location; or the first location and the second location can be in a same region of the intrathecal space.

In the above examples, the method can include creating an access opening into the intrathecal space of the patient in a lumbar region thereof and inserting a catheter through the access opening and threading the catheter rostrally within the intrathecal space, where infusion of the therapeutic bolus and the flush fluid is performed through the catheter. In one option, the catheter can include a first lumen with a first fluid port and a second lumen with a second fluid port, where the second fluid port is spaced proximally of the first fluid port, and threading the catheter rostrally within the intrathecal space positions the first fluid port at the first location within a lumbar region up to a cisterna magna region for infusion of the therapeutic bolus and positions the second fluid port at the second location within the lumbar region up to the cisterna magna for infusion of the flush fluid. Optionally, threading the catheter rostrally within the intrathecal space can position the first fluid port at the first location within a thoracic region up to the cervical region of the intrathecal space for infusion of the therapeutic bolus and can position the second fluid port at the second location within the lumbar region up to the cervical region of the intrathecal space for infusion of the flush fluid; and/or threading the catheter rostrally within the intrathecal spaced can include moving the first lumen relative to the second lumen within the intrathecal space. In another option, the catheter can include a single lumen with a distal fluid port, and threading the catheter rostrally within the intrathecal space can include positioning the distal fluid port at the first location within a lumbar region up to a cisterna magna region for infusion of the therapeutic bolus and withdrawing the catheter to position the distal fluid port at the second location within the lumbar region up to the cisterna magna for infusion of the flush fluid. Optionally, threading the catheter rostrally within the intrathecal space can include positioning the distal fluid port at the first location within a thoracic region up to a cervical region for infusion of the therapeutic bolus and withdrawing the catheter to position the distal fluid port at the second location within the lumbar region for infusion of the flush fluid.

In some of the above examples, the method can include creating an access opening into the intrathecal space of the patient in a lumbar region thereof with an introducer needle and inserting a catheter through the access opening and threading the catheter rostrally within the intrathecal space, where infusion of the therapeutic bolus is performed through the introducer needle and the infusion of the flush fluid is performed through the catheter.

In some of the above examples, the method can include creating an access opening into the intrathecal space of the patient in a lumbar region thereof with a lumbar puncture needle, where infusion of the therapeutic bolus and the flush fluid is performed through the lumbar puncture needle in the lumbar region.

In the above examples, the method can include one or more of the following options: infusing the flush fluid can include infusing an artificial cerebrospinal fluid or anti-inflammatory fluid into the intrathecal space of a patient in the second location at the one or more flush flow rates; the method can include positioning the patient in a head up tilt orientation prior to infusing the therapeutic bolus and infusing the flush fluid; the method can include positioning the patient in a head down tilt orientation prior to infusing the therapeutic bolus and infusing the flush fluid and the therapeutic bolus can include a buffer that is at least one of: colder than a temperature of cerebrospinal fluid of the patient; or more dense than the cerebrospinal fluid of the patient; at least one of the therapeutic bolus or the flush fluid can include a contrast agent; the therapeutic bolus can include a nucleic acid, a protein therapeutic, a cell therapy, a small molecule therapeutic, a viral vector encoding a therapeutic protein, or a combination thereof, where, optionally, the therapeutic bolus includes the nucleic acid selected from the group consisting of an antisense oligonucleotide, a ribozyme, an miRNA, an siRNA, and shRNA, or a nucleic acid encoding a clustered regularly interspaced short palindromic repeats (CRISPR) associated protein (Cas) system, or a combination thereof, or the therapeutic bolus includes an antisense oligonucleotide that targets mRNA encoding Huntington protein (HTT) or an antisense oligonucleotide that targets mRNA encoding survival motor neuron-2 (SMN2); the therapeutic bolus can include adeno-associated virus (AAV) vector DNA sequences, recombinant AAV particles, or combinations thereof, where, optionally, the AAV vector DNA sequences target mRNA encoding a MECP2 gene; or the therapeutic bolus can be infused according to a therapy platform comprising one of: conventional gene replacement, x-reactivation, exon skipping, or promoter modulation.

In accordance with a second example, a method for fluid delivery to a target area in a central nervous system of a patient is described that includes infusing a therapeutic bolus into an intrathecal space of a patient in a first location and subsequently infusing a flush fluid into the intrathecal space of the patient in a second location at one or more flush flow rates. A volume of the flush fluid corresponds to a volume of cerebrospinal fluid (CSF) between the first location. Optionally, the method can include imaging the patient to determine the volume of the cerebrospinal fluid between the first location and the target area.

In the above examples, the one or more flush flow rates can be based on at least one of patient anatomy or physiology data. Optionally, the one or more flow rates can be calculated using estimated or measured steady-streaming fluid velocities of cerebrospinal fluid within the intrathecal space of the patient between the first location and the target area divided into one or more axial sections and estimated or measured axial cross-sectional areas of the one or more axial sections. Optionally, the one or more flush flow rates can be based on averages of the steady-streaming fluid velocities or maximum values of the steady-streaming fluid velocities in the one or more axial sections and, further, if desired, the one or more flush flow rates can be calculated using a predetermined percentage of the averages of the steady-streaming fluid velocities or the maximum values of the steady-streaming fluid velocities in the one or more axial sections. Optionally, the at least one of patient anatomy or physiology data can include data obtained from patient imaging and tests, and computations performed on the patient imaging and tests; Optionally, the at least one of patient anatomy or physiology data can include one or more of: patient age, patient sex, patient size, patient CSF volume, patient CSF dynamics, patient respiration data, patient sleep data, patient anatomical geography, heart rate, or disease. Further, if desired, the steady-streaming fluid velocities can be estimated by a central nervous system computational or in vitro model for the patient using the at least one of patient anatomy or physiology data as input.

In the above examples, the method can include one or more of the following: the target area can be the brain, the spine or combinations thereof; the method can include measuring CSF pressure of the patient with a pressure sensor and stopping the infusion of the flush fluid or reducing the one or more flush flow rates in response to determining that the CSF pressure exceeds a predetermined threshold or drops below a predetermined threshold; the method can include measuring CSF pressure of the patient with a pressure sensor and beginning the infusion of the flush fluid in response to determining that the CSF pressure indicates an ascending or descending phase of a waveform; the second location can be spaced caudally from the first location; or the first location and the second location can be in a same region of the intrathecal space.

In the above examples, the method can include creating an access opening into the intrathecal space of the patient in a lumbar region thereof and inserting a catheter through the access opening and threading the catheter rostrally within the intrathecal space, wherein infusion of the therapeutic bolus and the flush fluid is performed through the catheter. Optionally, the catheter can include a first lumen with a first fluid port and a second lumen with a second fluid port, where the second fluid port is spaced proximally of the first fluid port, and threading the catheter rostrally within the intrathecal space positions the first fluid port at the first location within a lumbar region up to a cisterna magna region for infusion of the therapeutic bolus and positions the second fluid port at the second location within the lumbar region up to the cisterna magna for infusion of the flush fluid. Optionally, threading the catheter rostrally within the intrathecal space can position the first fluid port at the first location within a thoracic region up to the cervical region of the intrathecal space for infusion of the therapeutic bolus and position the second fluid port at the second location within the lumbar region up to the cervical region of the intrathecal space for infusion of the flush fluid. Optionally, threading the catheter rostrally within the intrathecal spaced can include moving the first lumen relative to the second lumen within the intrathecal space. In another option, the catheter can include a single lumen with a distal fluid port, and threading the catheter rostrally within the intrathecal space can include positioning the distal fluid port at the first location within a lumbar region up to a cisterna magna region for infusion of the therapeutic bolus and withdrawing the catheter to position the distal fluid port at the second location within the lumbar region up to the cisterna magna for infusion of the flush fluid. Optionally, threading the catheter rostrally within the intrathecal space can include positioning the distal fluid port at the first location within a thoracic region up to a cervical region for infusion of the therapeutic bolus and withdrawing the catheter to position the distal fluid port at the second location within the lumbar region for infusion of the flush fluid.

In some of the above examples, the method can include creating an access opening into the intrathecal space of the patient in a lumbar region thereof with an introducer needle and inserting a catheter through the access opening and threading the catheter rostrally within the intrathecal space, where infusion of the therapeutic bolus is performed through the introducer needle and the infusion of the flush fluid is performed through the catheter.

In some of the above examples, the method can include creating an access opening into the intrathecal space of the patient in a lumbar region thereof with a lumbar puncture needle, where infusion of the therapeutic bolus and the flush fluid is performed through the lumbar puncture needle in the lumbar region.

In the above examples, the method can include one or more of the following: infusing the flush fluid can include infusing an artificial cerebrospinal fluid or anti-inflammatory fluid into the intrathecal space of a patient in the second location at the one or more flush flow rates; the method can include positioning the patient in a head up tilt orientation prior to infusing the therapeutic bolus and infusing the flush fluid; the method can include positioning the patient in a head down tilt orientation prior to infusing the therapeutic bolus and infusing the flush fluid and the therapeutic bolus can include a buffer being at least one of: colder than a temperature of cerebrospinal fluid of the patient; or more dense than the cerebrospinal fluid of the patient; at least one of the therapeutic bolus or the flush fluid can include a contrast agent; the therapeutic bolus can include a nucleic acid, a protein therapeutic, a cell therapy, a small molecule therapeutic, a viral vector encoding a therapeutic protein, or a combination thereof; the therapeutic bolus can include the nucleic acid selected from the group consisting of an antisense oligonucleotide, a ribozyme, an miRNA, an siRNA, and shRNA, or a nucleic acid encoding a clustered regularly interspaced short palindromic repeats (CRISPR) associated protein (Cas) system, or a combination thereof, where, optionally, the therapeutic bolus can include an antisense oligonucleotide that targets mRNA encoding Huntington protein (HTT) or an antisense oligonucleotide that targets mRNA encoding survival motor neuron-2 (SMN2); the therapeutic bolus can include adeno-associated virus (AAV) vector DNA sequences, recombinant AAV particles, or combinations thereof, where, optionally, the AAV vector DNA sequences can target mRNA encoding a MECP2 gene; or the therapeutic bolus can be infused according to a therapy platform comprising one of: conventional gene replacement, x-reactivation, exon skipping, or promoter modulation.

In accordance with a third example, a fluid delivery system is described that includes a pump device including a first syringe configured to contain a therapeutic bolus, a second syringe configured to contain a flush fluid, and one or more drivers configured to cause the therapeutic bolus and the flush fluid to be expelled from the first and second syringes, respectively. The fluid delivery system further includes a fluid delivery device fluidly coupled to the plurality of syringes and a controller. The controller is configured to control operation of the pump device to expel the therapeutic bolus from the first syringe and deliver the therapeutic bolus to the fluid delivery device for a first infusion into an intrathecal space of a patient in a first location and subsequently control operation of the pump device to expel the flush fluid from the second syringe and deliver flush fluid to the fluid delivery device at one or more flush flow rates for a second infusion into the intrathecal space of the patient in a second location, where the one or more flush flow rates are based on at least one of patient anatomy or physiology. Optionally, the first location can be within a lumbar region up to a cistern magna region of the patient for infusion of the therapeutic bolus and the second location can be within a lumbar region up to a cisterna magna region of the patient for infusion of the flush fluid. If desired, the first location can be within a thoracic region up to the cervical region and the second location can be within the lumbar region up to a cervical region.

In the above examples, the fluid delivery device can include a catheter, where the catheter includes a first lumen with a first fluid port, where the first lumen is sized to position the first fluid port at the first location, and a second lumen with a second fluid port, where the second lumen is sized to position the second fluid port at the second location. Optionally, the first lumen can be movably received within the second lumen. Optionally, the system can include a valve that is configured to allow a length of the first lumen to be adjusted relative to the second lumen while maintaining a fluid seal of the second lumen.

In some of the above examples, the fluid delivery system can include an introducer needle fluidly coupled to the plurality of syringes, where the introducer needle includes a lumen with a fluid port, and a catheter fluidly coupled to the plurality of syringes and configured to be threaded through the lumen of the introducer needle, where the catheter includes a lumen with a fluid port. The controller can then be configured to control operation of the pump device to expel the therapeutic bolus from the first syringe and deliver the therapeutic bolus to the catheter for the first infusion through the fluid port of the catheter into the intrathecal space of the patient in the first location and subsequently control operation of the pump device to expel the flush fluid from the second syringe and deliver flush fluid to the introducer needle at the one or more flush flow rates for the second infusion through the fluid port of the introducer needle into the intrathecal space of the patient in the second location.

In the above examples, the fluid delivery system can include one or more of the following: the catheter can include an atraumatic tip; the second location can be spaced caudally from the first location; the first location and the second location can be in a same region of the intrathecal space; the controller can be internal to the pump device; the system can include a display configured to provide a user interface.

In some of the above examples, the fluid delivery device can include a lumbar puncture needle fluidly coupled to the plurality of syringes, where the lumbar puncture needle includes a lumen with a fluid port. The controller can then be configured to control operation of the pump device to expel the therapeutic bolus from the first syringe and deliver the therapeutic bolus to the lumbar puncture needle for the first infusion through the fluid port of the lumbar puncture needle into the intrathecal space of the patient in the first location and subsequently control operation of the pump device to expel the flush fluid from the second syringe and deliver flush fluid to the lumbar puncture needle at the one or more flush flow rates for the second infusion through the fluid port of the lumbar puncture needle into the intrathecal space of the patient in the second location

In the above examples, the fluid delivery system can include a pressure sensor that is configured to measure cerebrospinal fluid (CSF) pressure of the patient and the controller can be in communication with the pressure sensor and configured to at least one of: stop the first infusion or the second infusion in response to determining that the CSF pressure exceeds a predetermined threshold, reduce the one or more flush flow rates in response to determining that the CSF pressure exceeds a predetermined threshold, or begin the second infusion in response to determining that the CSF pressure indicates an ascending or descending phase of a waveform. Optionally, the pressure sensor can be in line or coupled to the catheter along the length thereof to be positioned within the intrathecal space of the patient.

In the above examples, the one or more flow rates can be calculated using estimated or measured steady-streaming fluid velocities of cerebrospinal fluid within the intrathecal space of the patient between the first location and the target area divided into one or more axial sections and estimated or measured axial cross-sectional areas of the one or more axial sections. Optionally, the one or more flush flow rates can be based on averages of the steady-streaming fluid velocities or maximum values of the steady-streaming fluid velocities in the one or more axial sections. Further, if desired, the one or more flush flow rates can be calculated using a predetermined percentage of the averages of the steady-streaming fluid velocities or the maximum values of the steady-streaming fluid velocities in the one or more axial sections. Optionally, the at least one of patient anatomy or physiology data can include data obtained from patient imaging and tests, and computations performed on the patient imaging and tests; and/or patient age, patient sex, patient size, patient CSF volume, patient CSF dynamics, patient respiration data, patient sleep data, patient anatomical geography, heart rate, or disease. Optionally, the steady-streaming fluid velocities can be estimated by a central nervous system computational or in vitro model for the patient using the at least one of patient anatomy or physiology data as input.

In the above examples, the fluid delivery system can include one or more of the following: the target area can be the brain, the spine or combinations thereof; the flush fluid can include an artificial cerebrospinal fluid or anti-inflammatory fluid; the therapeutic bolus can include a buffer being at least one of: colder than a temperature of cerebrospinal fluid of the patient; or more dense than the cerebrospinal fluid of the patient; at least one of the therapeutic bolus or the flush fluid can include a contrast agent; the therapeutic bolus comprises a nucleic acid, a protein therapeutic, a cell therapy, a small molecule therapeutic, a viral vector encoding a therapeutic protein, or a combination thereof, where, optionally, therapeutic bolus includes the nucleic acid selected from the group consisting of an antisense oligonucleotide, a ribozyme, an miRNA, an siRNA, and shRNA, or a nucleic acid encoding a clustered regularly interspaced short palindromic repeats (CRISPR) associated protein (Cas) system, or a combination thereof or the therapeutic bolus includes an antisense oligonucleotide that targets mRNA encoding Huntington protein (HTT) or an antisense oligonucleotide that targets mRNA encoding survival motor neuron-2 (SMN2); the therapeutic bolus can include adeno-associated virus (AAV) vector DNA sequences, recombinant AAV particles, or combinations thereof, where, optionally, the AAV vector DNA sequences target mRNA encoding a MECP2 gene; or the therapeutic bolus can be infused according to a therapy platform comprising one of: conventional gene replacement, x-reactivation, exon skipping, or promoter modulation.

In accordance with a fourth example, a catheter is described that includes a first lumen with a first fluid port and a second lumen with a second fluid port, where the second lumen has an annular configuration with a distal opening sized to telescopingly receive the first lumen therethrough. Optionally, distal tips of the first and second lumens can have atraumatic configurations; the catheter can include a pressure sensor coupled along the length thereof to be positioned within a patient during use of the catheter; and/or the catheter can include a valve configured to allow a length of the first lumen to be adjusted relative to the second lumen while maintaining a fluid seal of the second lumen.

### BRIEF DESCRIPTION OF THE DRAWINGS

FIG. 1 is a diagrammatic view of an example fluid delivery system in accordance with various embodiments;

FIG. 2 is a sectional view of an example pump device for a fluid delivery system in accordance with various embodiments;

FIG. 3 is a sectional view of a distal tip of a catheter for a fluid delivery system in accordance with various embodiments;

FIG. 4 is a sectional view of a catheter having first and second lumens in telescoping relation relative to one another in accordance with various embodiments;

FIG. 5 is a sectional view of a catheter having first and second lumens in side-by-side relation in accordance with various embodiments;

FIG. 6 is a sectional view of a needle having a lumen and a catheter threaded through the lumen of the needle in accordance with various embodiments;

FIG. 7 is a sectional view of a lumbar puncture needle and portion of an intrathecal space in accordance with various embodiments;

FIG. 8 is a perspective view of a plug-in port for mounting to a bone of a patient in accordance with various embodiments;

FIG. 9 is a schematic view of the plug-in port of FIG. 8 mounted to bone to access an intrathecal space;

FIG. 10 is a diagrammatic view of a central nervous system axial section to determine an infusion flow rate based on average steady-streaming fluid velocities in accordance with various embodiments;

FIG. 11 is a diagrammatic view of a central nervous system axial section to determine an infusion flow rate based on maximum steady-streaming velocities in accordance with various embodiments;

FIG. 12 is a diagrammatic view of a central nervous system showing example cross-sectional drug mass after a lumbar puncture infusion; and

FIG. 13 is a diagrammatic view of a central nervous system showing example cross-sectional drug mass after an algorithmic infusion according to various embodiments.

### DETAILED DESCRIPTION

The systems and methods described herein are configurable central nervous system (CNS) delivery solutions for therapeutics, such as genetic medicines. In addition to a therapeutic bolus delivered within intrathecal space, the systems and methods described herein utilize a flush, which can optionally be performed caudally to the delivery location of the therapeutic bolus, to move the therapeutic bolus rostrally toward a target area, such as the brain, spine, or both, and achieve a desired spread in the spine and/or brain. Advantageously, the configuration of the flush and the delivery location can be configured to deliver a therapeutic dose to the brain greater than 100 times the typical intrathecal drug delivery via lumbar puncture and reduce drug dose to the spinal cord by 100 times compared to lumbar puncture. The flush can also be configured allow a greater amount of the therapeutic bolus to spread in the spine and brain as it travels rostrally from the delivery location, or to deliver the therapeutic bolus entirely to the spine by limiting the extent of the flush or location (caudal versus rostral, or vice versa). Accordingly, the systems and methods are customizable for different therapies to deliver a drug or other therapeutic to desired areas of the spine and/or brain and in predetermined quantities. The systems and methods can be customized to be patient or group specific and can be customized for a particular therapy. For example, the fluid delivery device location and infusion can be based on one or more of patient age, patient sex, patient size, patient CSF volume, patient CSF dynamics and physiologic parameters such as respiration or sleep, patient anatomical geography, heart rate, or disease.

The systems and methods can include a first infusion of a therapeutic bolus in a first location from the lumbar region to the cisterna magna region followed by a second infusion of a flush fluid in a second location from the lumbar region to the cisterna magna region. In one example, the second infusion can be performed at one or more flush flow rates that are based on at least one of patient anatomy or physiology data. In another example, the second infusion can be of a volume corresponding to a CSF volume between a target area for the therapeutic bolus and the first location.

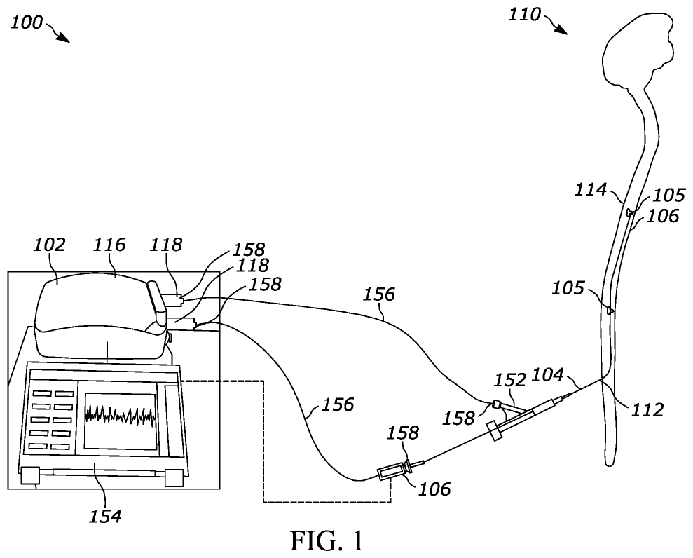

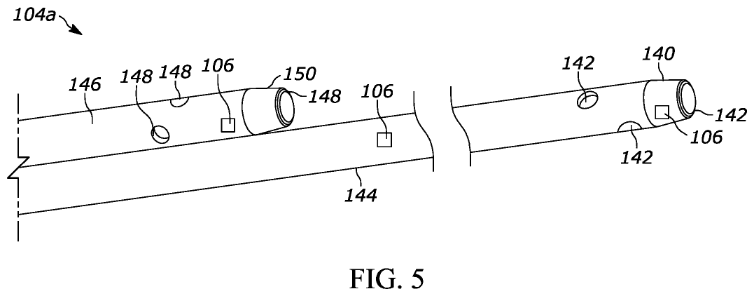

An example fluid delivery system 100 suitable for the methods of treatment described herein is shown in FIGS. 1-5. The example system 100 includes a pump device 102, a fluid delivery device 104, such as a catheter and/or needle, optionally a pressure sensor 106, and a controller 108 configured to control infusion through the fluid delivery device 104 by operation of the pump device 102. In some examples, the controller 108 can further be configured to monitor CSF pressure, such as intrathecal and intracranial pressure, during infusion based on data from the optional pressure sensor 106. The controller 108 can be a component of the pump device 102 or a separate component, as desired.

FIG. 1 illustrates an example of a pump device 102 constructed in accordance with the teachings of the present disclosure and positioned in proximity to a patient 110 to perform an infusion of fluid through an access location 112 in the patient 110. For example, the access location 112 can be a lumbar puncture to access an intrathecal space 114 of the patient 110. Once the pump device 102 is fluidly coupled to the intrathecal space 114 via the catheter 104, the clinician may activate the pump device 102 to begin delivery of a fluid (i.e., infusion) to the intrathecal space 114. In particular, once activated, the pump device 102 delivers a fluid to the intrathecal space 114 based on a programmed algorithmic infusion profile, discussed in more detail below. It will be appreciated that the pump device 102 may be programmed with an algorithmic infusion developed off-site or on-site.

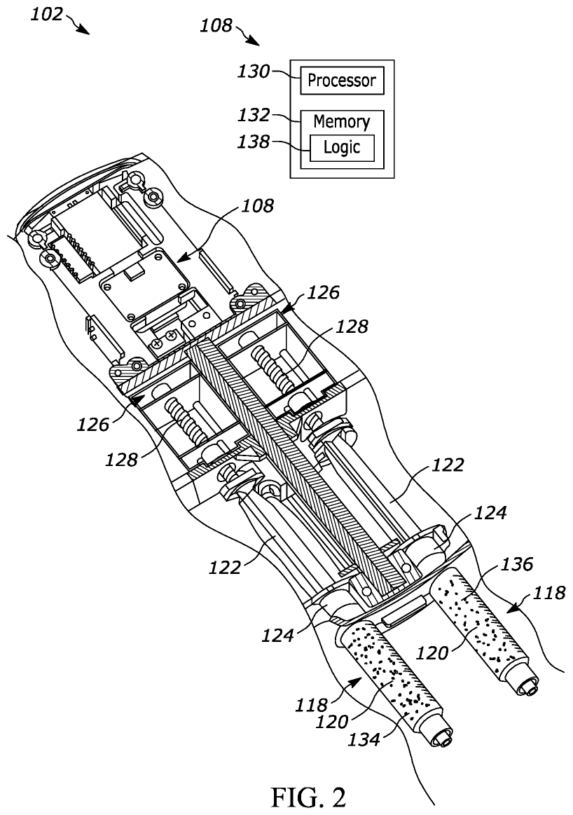

FIG. 2 shows further details regarding an example pump device 102. The pump device 102 in this example generally includes a housing 116, a plurality of syringes 118 carried by (e.g., partially disposed within) the housing 116 and adapted to be fluidly coupled to the intrathecal space 114. In this example, the housing 116 can have a table-top or hand-held configuration, as desired.

In the illustrated example, the pump device 102 can include two syringes 118 carried by the housing 116 and partially disposed therein. Of course, it will be understood that the plurality of syringes 118 may include more than two syringes 118 (e.g., three or four syringes 118). In any case, each one of the plurality of syringes 118 includes a barrel 120 partially disposed within the housing 116 and adapted to hold a fluid and/or receive a fluid depending on an infusion or aspiration action, a plunger rod 122 movably disposed within the barrel 120, and a stopper 124 adjacent or mounted to a proximal end of the plunger rod 122 and disposed within the barrel 120. Coupled to the plurality of syringes 118 is at least one actuator 126 that is disposed within the housing 116. The pump device 102 can include a plurality of actuators 126, one coupled to each of the plurality of syringes 118, such that, in the illustrated example, two actuators 126 are employed. However, in other examples, more or fewer actuators 126 may be employed. Each actuator 126 can include a driver 128 operably coupled to the plunger rod 122 to drive movement thereof via a connecting structure, such as a shaft, gear train, etc. In particular, the drivers 128 control the position of the plunger rods 122 and, in turn, the flow of fluid to and/or from the intrathecal space 114, by translating the plunger rods 122 and stoppers 124 toward a proximal end of the respective barrel 120 or away from the proximal end of the respective barrel 120.

As discussed above, the system 100 also includes a controller 108 to control operation of the pump device 102 and, optionally, monitor data from the pressure sensor 106. The controller 108 can be disposed in, e.g., internal to, the housing 116 or can be external thereto and in communication with the pump device 102. The controller 108 is communicatively coupled to the actuators 126 and controls the position of actuators 126, which ultimately controls the movement of the plunger rods 122 to infuse a fluid into the intrathecal space 114 and/or aspirate the fluid from the intrathecal space 114.

The controller 108 includes a processor 130 that implements the algorithmic infusion stored in a memory 132 of the controller 108. The algorithmic infusion stored in the memory 132 includes an infusion protocol for at least one of the syringes 118. Generally speaking, the processor 130 communicates with the actuators 126 to execute the algorithmic infusion. For example, the controller 108 can be communicatively coupled to the actuators 126 using a hardwired communication scheme which may include the use of any desired hardware, software and/or firmware to implement hardwired communications, including, for example, standard 4-20 mA communications, and/or any communications using any smart communication protocol such as the FOUNDATION® Fieldbus communication protocol, the HART® communication protocol, RS-485, RS-232, etc. In another example, the controller 108 can be communicatively coupled to the actuators 126 using a wireless communication scheme facilitated using the WirelessHART® protocol, the Ember protocol, a WiFi protocol, and IEEE wireless standard, etc.

The processor 130 may be a general processor, a digital signal processor, ASIC, field programmable gate array, graphics processing unit, analog circuit, digital circuit, or any other known or later developed processor. The processor 130 operates pursuant to the algorithmic infusion stored in the memory 132. The memory 132 may be a volatile memory or a non-volatile memory. The memory 132 may include one or more of a read-only memory (“ROM”), random-access memory (“RAM”), a flash memory, an electronic erasable program read-only memory (“EEPROM”), or other type of memory. The memory 132 may include an optical, magnetic (hard drive), or any other form of data storage.

The algorithmic infusion is a set of executable instructions that controls at least one of the syringes 118 to facilitate infusion of a plurality of fluids 134, 136 into the patient 110 using the pump device 102. The algorithmic infusion may be stored on the memory 132 as computing logic 138, which includes one or more infusion and aspiration routines and/or sub-routines, embodied as computer-readable instructions stored on the memory 132. The controller 108, particularly the processor 130 thereof, can execute the logic 138 to cause the processor 130 to retrieve the algorithmic infusion and control the actuators 126 in accordance with the algorithmic infusion in order to facilitate the desired infusion and/or aspiration of the particular fluid 134, 136 into the patient 110. In particular, the algorithmic infusion may specify, among other parameters, whether each of the syringes 118 is to infuse the fluid 134, 136 into the intrathecal space 114 (or aspirate the fluid 134, 136 from the intrathecal space 114), the timing of infusion and/or aspiration (i.e., when the plunger rods 122 are to be moved), a volume of the fluid 134, 136 to be infused into the intrathecal space 114 from the barrel(s) 120, the flow rate for infusing the fluid 134, 136 into the intrathecal space 114 from the barrel(s) 120, a volume of the fluid 134, 136 to be aspirated from the intrathecal space 114 into the barrel(s) 120, the flow rate for aspirating the fluid 134, 136 from the intrathecal space 114 into the barrel(s) 120.

In some cases, the algorithmic infusion may be stored on a memory outside of the controller 108 and transmitted to the controller 108 prior to operation of the pump device 102. For example, the algorithmic infusion can be stored on a memory of a desktop computer which communicates with the controller 108 wirelessly or through a hardwired connection using any of the wireless communication or hardwired communication protocols discussed above. In other examples, the infusion and aspiration profile can be stored on a memory of a mobile electronic device, a smart phone, or a server located away from the controller 108. Additionally, the algorithmic infusion may be stored on an external memory and transferred to the memory 132 of the controller 108 through a hardwired connection. For example, the algorithmic infusion can be stored on an external hard drive, a solid-state drive (“SSD”), a portable digital storage device, the Cloud, a Personal Cloud, or a USB Flash Drive, and then transferred to the memory 132.

An example handheld auto-injection pump device is disclosed in U.S. Pat. No. 10,675,438, which is hereby incorporated by reference herein in its entirety.

Example fluid delivery devices 104 suitable for the systems and methods described herein are shown in FIGS. 1 and 3-6. As described in more detail below, an example algorithmic infusion can include a first infusion of a first fluid at a first location and a second infusion of a second fluid at a second location that is caudal of the first location. In one example, the first location can range from the lumbar region to the cisterna magna region of a patient. In another example, the first location can range from the thoracic region to the cervical region of a patient. In one example, the second location can range from the lumbar region to the cisterna magna region of a patient. In another example, the second location can be within the lumbar region to the cervical region of a patient.

In some examples, the fluid delivery device 104 can be a catheter 104a. In these examples, the catheter 104a is threadable and can include an atraumatic tip 140 to implant the catheter 104a within the intrathecal space 114 and thread the tip 140 to a desired location along the spine. For example, the tip 140 can have a tapered configuration, e.g. frustoconical with a distal port, or a curved configuration for atraumatic threading. The catheter 104a includes one or more fluid ports 142 at the tip 140 thereof to dispense fluid rostrally within the intrathecal space 114. The fluid ports 142 can include a distal port extending axially through the tip 140 and/or one or more side ports extending through a sidewall of the catheter 104a, as desired.

In a first example form, the catheter 104a can include a single lumen 144 with the fluid port 142 at the tip 140 of the catheter 104a. In this form, a clinician can thread the catheter 104a to place the catheter tip 140 at the first location, infuse the first fluid, withdraw the catheter 104a to place the catheter tip 140 at the second location, and infuse the second fluid.

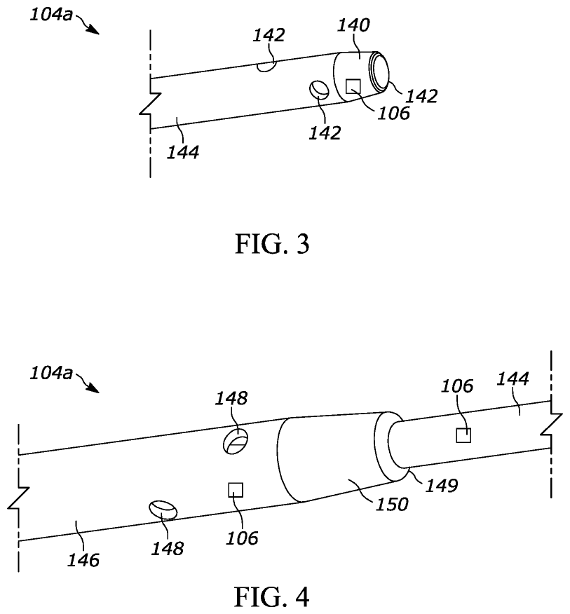

In other forms as shown in FIGS. 3-5, the catheter 104a can include a second lumen 146 with a second fluid port 148 spaced rearwardly along the length of the catheter 104a from the tip 140. The first and second lumens 144, 146 can be side-by-side or concentric, e.g., the first lumen 144 extending within the second lumen 146, as desired. Further, the first and second lumens 144, 146 and fluid ports 142, 148 thereof can be fixed in relation to one another. Alternatively, the first and second lumens 144, 146 can be moveable with respect to one another. For example, as shown in FIG. 4, the second lumen 146 can have an annular configuration with a distal outlet 149 sized to receive the first lumen 144 therethrough, such that the first lumen 144 can telescope relative to the second lumen 146. The advantage of movable lumens 144, 146 is that the catheter 104a can adapt to different patient-specific intrathecal lengths to position the first and second ports 142, 148 in desired locations.

As shown in FIGS. 3-5, with a dual lumen catheter 104a, the second lumen 146 can have an associated atraumatic tip 150. For example, the second tip 150 can also have a tapered configuration, e.g. frustoconical with a distal port and/or the inner lumen 144 extending therethrough, or a curved configuration for atraumatic threading.

For catheters 104a having lumens 144, 146 that are movable relative to one another, the system 100 can include a valve 152 (FIG. 1) which allows the length of the first or inner lumen 144 to be adjusted within the intrathecal space relative to the second or outer lumen 146 while maintaining a fluid seal of the second lumen 146. For example, the valve 152 can be a Touhy Borst hemostasis valve that threads the first or inner lumen 144 into the second or outer lumen 146.

In some forms, one or both lumens 114, 146 can be lined with one or more suitable materials to mitigate adhesion of a particular drug or other fluid to be dispensed therethrough. For example, for a lumen 114, 116 intended to dispense AAV, the lumen 114, 116 can be made from, include an inner layer of, or be lined with polytetrafluoroethylene (PTFE) to mitigate AAV adhesion. Other suitable compatible materials can alternatively be utilized.

In one example, overall useable length of the catheter 104a can be between 12 inches and 45 inches with the inner lumen length adjustable from 0″ to 6.3″ (16 cm). One example, the inner lumen can be 0.0155″ to fit a 0.14 guidewire or smaller for 0.010 guidewire.

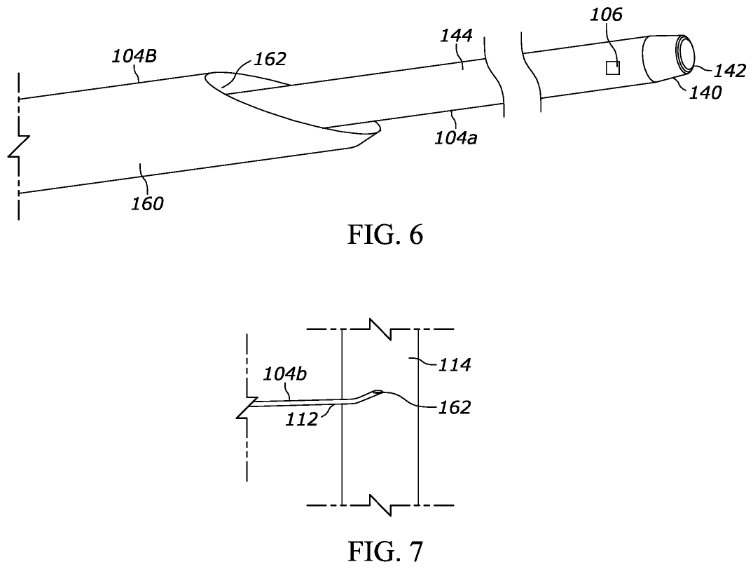

In one example, as shown in FIG. 6, the fluid delivery device 104 can also include an introducer needle 104b configured to introduce the catheter 104a to the intrathecal space 114 through the access location 112. The introducer needle 104b includes a lumen 160 through which the catheter 104a can be threaded to place the distal tip(s) 140, 150 in a desired location with the intrathecal space 114. In some examples, the introducer needle 104b can be utilized to infuse the first fluid and/or the second fluid. For example, a distal port 162 of the needle lumen 160 can be utilized to infuse the flush fluid in the second location as discussed herein.

In another example, as shown in FIG. 7, the fluid delivery device 104 can be a lumbar puncture needle 104b configured to be inserted through the access location 112 into the intrathecal space 114. With this configuration, a clinician can insert the needle 104b to position the port 162 to infuse the first fluid and subsequently infuse the second fluid. In this example, the first and second locations can be within the same region of the intrathecal space.

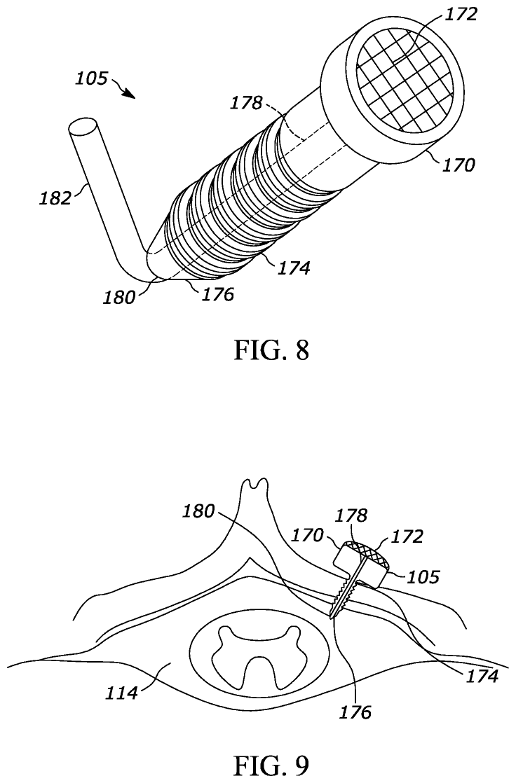

In another example as shown in FIGS. 8 and 9, the system 100 can include a plug-in port 105. The plug-in port 105 can be mounted for access to any desired CNS location, including in the lumbar region, the thoracic region, the cervical region, or intracranial to intracerebroventricular (ICV) or intra-cisterna magna (ICM). The plug-in port 105 can include a head portion 170 with one septum 170 or a plurality of septums 172, such as two, three, or four, coupled thereto. The septum 172 is configured to receive a needle 104b therethrough for the introduction of fluids, such as the therapeutic bolus or flush fluid as described herein, while also preventing fluid flow through the port 105 from the CNS location. The plug-in port 105 can be subcutaneous and accessible through the skin of the patient or can have the septum(s) exposed through the skin of the patient.

As shown, the plug-in port 105 can include an anchor portion 174 extending from the head portion 170 allowing the port 105 to be mounted to bone, such as a vertebra, of a patient. The head portion 170 can have a larger radial dimension than the anchor portion 174 so that the head portion 170 includes outwardly projecting structure configured to abut the bone. For example, the head and anchor portions 170, 174 can be cylindrical as shown and the anchor portion 174 can have a smaller diameter than the head portion. A bore or conduit 178 extends through the port 105 along the longitudinal length thereof with a first end adjacent to the septum 172 and an opposite, second end providing one or more fluid outlets 180 through the anchor portion 174. The fluid outlet 180 can be oriented to dispense fluid parallel with the longitudinal axis, as shown, and/or can extend radially through the anchor portion 174.

In one form, the anchor portion 174 can be sized to be inserted through the bone so that a distal tip 176 thereof is inserted into a desired CNS location, such as through the dura into the intrathecal space 114 or other CNS location. In another example, the port 105 can further include a flexible catheter or cannula 182 extending from the fluid outlet 180 and fluidly connected to the conduit 178. In this form, the catheter 182 can be inserted through the dura and can be threaded in a desired direction to direct fluid flow in a desired path within the intrathecal space 114 or other CNS location. In another example, a clinician can insert the needle 104b entirely through the port 105 to directly access the intrathecal space 114 or other CNS location with the needle 104b. With any of these configurations, a clinician can insert a needle 104b through the septum 172 to access the conduit 178 and dispense a desired amount of fluid, such as the therapeutic bolus and/or flush fluid, which is then distributed into the intrathecal space 114 or other CNS location. The port 105 allows a clinician to have repeatable and reliable access to a desired CNS location.

In one example, the anchor portion 174 can have a screw or drill configuration, as shown in FIG. 8, such that a clinician can rotate the port 105 during installation to screw the port 105 into the bone and secure the port 105 in place. In another example, rather than a screw anchor portion 174, the port 105 can be fixed to the bone over a burr hole extending through the bone using screws or other fixation members and the anchor portion 174 can telescope from the head portion 170 through the burr hole to insert the tip 176 into the intrathecal space or other CNS location. Telescoping of the anchor portion 174 can be driven by the needle 104b, for example.

As shown in FIGS. 1, 3, and 4, the pressure sensor 106 can be provided in line between the pump device 102 and the catheter 104 or embedded within or mounted to the catheter 140a. For example, the pressure sensor 106 can be disposed along a useable length of the catheter 104a that will be disposed within the intrathecal space 114 to thereby measure CSF pressure, such as at the tip 140, 150. With any configuration, the pressure sensor 106 can provide CSF pressure data to the controller 108. The controller 108 can then monitor the pressure data to monitor for safety, as well as maintain intracranial pressure (ICP) within a desired therapeutic range (e.g., 5-15 mmHg) which can impact CSF outflow pathways, e.g., nerve root sleeves, cribuform plate, olfactory nerves, inner ear canal, and arachnoid granulation a. For example, controlling intracranial pressure (ICP) within the desired therapeutic range can enhance perfusion of a therapeutic bolus to the brain/CNS tissue while also limiting leakage via undesired routes. This is because ICP above a certain amount can increase leakage through the outflow pathways and reduce penetration of the therapeutic bolus into the CNS tissue. Accordingly, the controller 108 can be configured to stop infusion or reduce a flow rate of infusion of the first and/or second fluid in response to determining that the CSF pressure data exceeds or drops below the therapeutic range. In addition or alternatively, the controller 108 can be configured to monitor the CSF pressure data to identify waveforms and begin infusion of the first and/or second fluid along with ascending or descending phases of the waveform.

The pressure sensor 106 electronically communicates with the controller 108 using any known electronic communication methods. For example, the controller 108 may be communicatively connected to the sensor 106 using a hardwired communication scheme as described in detail above, using one or more known wireless communication protocols, or a combination thereof. In particular, communication between the controller 108 and the sensor 106 may be facilitated using the WirelessHART® protocol, the Ember protocol, a WiFi protocol, and IEEE wireless standard, etc. A protocol stack operation may be used by these communication protocols to receive, decode, route, encode and send wireless signals via an antenna to implement wireless communications between the controller 108 and the sensors 106.

Additionally, the pressure data may be stored in the memory 132. The controller 108 may also be communicatively coupled to an external computing device that could, for example, compare the measured pressure data to a threshold pressure to determine if the measured pressure data is within an acceptable, threshold range. For example, the external computing device could be a desktop computer, a tablet, a mobile phone, server, etc.

In some examples, the system 100 can include a display 154 (FIG. 1) to convey information to a clinician or other user regarding the infusions of the first and second fluids. The display 154 can be utilized to dynamically show the pressure data measured by the pressure sensor 106. The display 154 can also be a touch screen that facilitates interaction with the patient and the clinician through a user interface (“UI”). In particular, the UI may display the operational status of the pump device 102 (e.g., on, off, infusing, aspirating, infusing and aspirating, etc.) as well as receive input from the patient and/or the clinician. The UI may, for example, allow the clinician to start, stop, pause, or continue operation of the pump device 102. The UI may also allow the clinician to pre-program the pump device 102 prior to use thereof as well as receive other input from the clinician, such as, for example, modifications to the infusion and aspiration profile during operation of the pump device 102. The display 154 can be disposed on the housing 116 of the pump device 102 or can be a separate component, as desired.

As shown in FIG. 1, the system 100 can include any components needed to fluidly couple the pump device 102, and the syringes 118 thereof, with the lumens 144, 146 of the fluid delivery device 104 and, optionally, the valve 152. For example, the system 100 can include tubing 156, connectors 158, such as Leur connectors, and strain relief jackets or members for the various connections in the system 100.

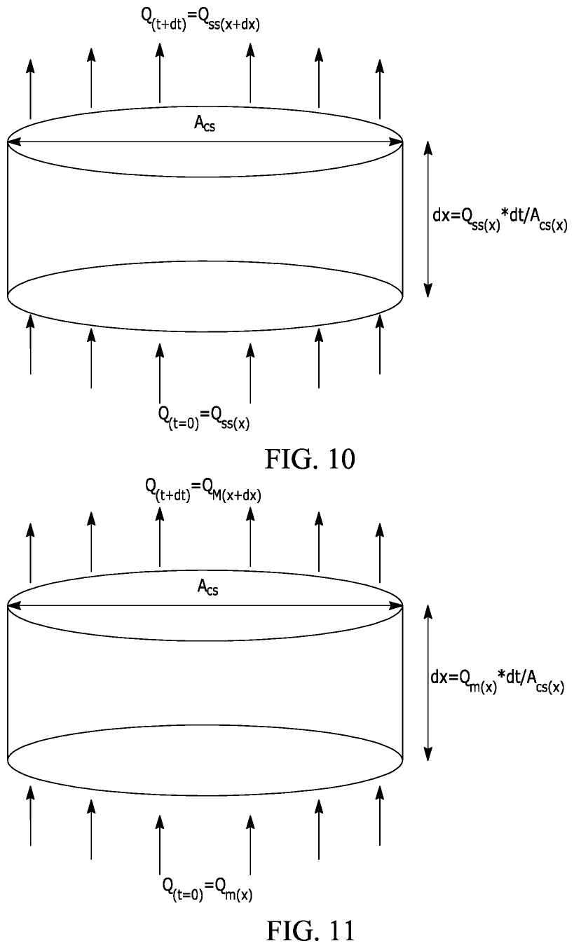

FIGS. 10 and 11 show portions of algorithmic processes for determining an infusion protocol for a therapeutic bolus followed by a flush fluid based on a location of a therapeutic bolus delivery within the intrathecal space 114, a therapeutic bolus delivery target area, and one or more input parameters. The input parameters can be one or more of: patient age, patient size, patient CSF volume, patient CSF dynamics, patient anatomical geography, optionally including nerve roots, physiologic parameters, such as heart rate, respiration, or sleep, patient anatomical geography, or disease, including stage or state of the disease. One or more of the input parameters can be estimated according to corresponding patient group data that the patient fits within or patient-specific obtained by imaging or other testing, including computations performed on the imaging or testing. By one approach, the aim of the algorithmic infusion is to get a predetermined percentage of the therapeutic bolus to the target area, such as the middle of the brain, specific areas of the brain, e.g., the right hemisphere, the left hemisphere, the ventricular system, the basal cistern, and/or the cerebellum, specific areas of the spine, or combinations thereof. The flush fluid can be infused into the intrathecal space 114 caudal to the location of the therapeutic bolus delivery a sufficient distance to ensure that the flush fluid pushes most or an entirety of the therapeutic bolus rostrally. Alternatively, the flush fluid can be infused in the same region or location of the intrathecal space 114 as the therapeutic bolus.

In one example, after the parameters are determined, a user can generate a CNS model for the patient with one of the CNS models known in the art with the parameters as input to estimate the CNS fluid dynamics for the patient and provide steady-streaming fluid velocities of the CSF within the intrathecal space 114. Using the steady-streaming fluid velocity data from the CNS model as input, the CNS can be divided into one or more axial sections depending on the level of precision desired for a particular infusion. For example, the length of the axial sections can range from a length of the therapeutic bolus delivery location to the target area, down to axial sections having a length of about 1 mm to about 750 mm in adults. For each axial section, the algorithm or can calculate/utilize an average steady-streaming fluid velocity, U_{SS}, for each axial section based on data from the CNS model.

Using an average cross-sectional area, A_{CS}, of each particular axial section, the algorithm can determine an average steady-streaming flow rate for the particular axial section, Q_{SS}, with the following equation:

Q_{SS} (ml/min)=U_{SS}*A_{CS}=(mm³/s)(60/1000)

Thereafter the algorithm can determine a flush flow rate at each time point during the algorithmic infusion based on flow movement from the therapeutic delivery port location along the spine. For example, the total flow movement (dx) at each time step (dt)=steady-streaming flow rate magnitude (Q_{SS}) divided by the cross-sectional area (A_{CS}).

As shown in FIG. 10, these equations can be reflected as:

Q₍ₜ₌₀₎=Q_{SS(x=port_location)};Q_{(t+dt)}=Q_{SS(x+dx)} where dx=Q_{SS(x)}*dt/A_{CS(x)}

With this configuration, the algorithmic infusion can dynamically control the flush flow rate as the therapeutic bolus is advanced within the CNS based on the location of the therapeutic bolus delivery within the intrathecal space 114 and the Q_{SS} at each location within the intrathecal space 114 until the therapeutic bolus reaches the predetermined target area.

In an alternative form, the algorithm can utilize a maximum value of the steady-stream fluid velocity, U_{M}, identified by the CNS model for each axial section. The maximum value shows the absolute value peak steady-streaming fluid velocity around the spinal cord at the particular axial section. The steady-streaming fluid velocity around the spinal cord is non-uniform at any axial section. If the algorithmic infusion imposes a flush rate that produces a velocity greater than the maximum caudal-directed velocity for each axial section, or a largest maximum velocity for all of the axial sections, no portion of the therapeutic bolus would move caudally down the spinal cord.

Using the average cross-sectional area, A_{CS}, of each particular axial section, the algorithm can determine a maximum flow rate for the particular axial section, Q_{M}, with the following equation:

Q_{M}(ml/min)=U_{M}*A_{CS} (mm³/s)(60/1000)

Thereafter the algorithm can determine a flush flow rate at each time point during the algorithmic infusion based on flow movement from the therapeutic delivery port location along the spine. For example, the total flow movement (dx) at each time step (dt)=streaming flow rate magnitude (Q_{M}) divided by the cross-sectional area (A_{CS}).

As shown in FIG. 11, these equations can be reflected as:

Q₍ₜ₌₀₎=Q_{M(x=port_location)};Q_{(t+dt)}=Q_{M(x+dx)} where dx=Q_{M(x)}*dt/A_{CS(x)}.

While the algorithmic infusion can utilize the flush flow rate(s) determined by the above steps, either based on the average steady-streaming fluid velocities or maximum values of the steady-streaming fluid velocities, the algorithmic infusion can alternatively operate at a predetermined percentage of the steady-steaming fluid velocities based on what percentage of the therapeutic bolus is desired to reach the target location and/or based on an ICP threshold value, e.g., between 100-105% of the chosen value, 50% of the chosen value, 75% of the chosen value, and so forth. If the chosen flush flow rate is lower than the maximum value(s), some percentage of the therapeutic bolus can potentially move caudally down the spine. In addition or as an alternative to a percentage of therapeutic bolus, a desired flush rate can be maintained until a CSF pressure threshold value is reached and then the flush flow rate can be lowered to maintain the CSF pressure below or at the threshold or within the threshold range.

With this configuration, the algorithmic infusion can dynamically control the flush flow rate as the therapeutic bolus is advanced within the CNS based on the location of the therapeutic bolus delivery within the intrathecal space 114 and the Q_{M} at each location within the intrathecal space 114 until the therapeutic bolus reaches the predetermined target area, such as the middle of the brain. The algorithmic infusion can be tailored as desired to ensure that a high percentage, e.g., 80-95%, of the therapeutic bolus is delivered to the brain (i.e., a rostral focused delivery) or a lower percentage, e.g., between 5-20%, of the therapeutic bolus is delivered to the brain, which allows the remaining percentage to spread in the spine and brain as the therapeutic bolus travels rostrally from the delivery location. In fact, the spread of the therapeutic bolus can be limited entirely to the spine by only flushing the therapeutic bolus up to a cervical location.

Additionally, the total amount of flush fluid to be infused, i.e., the flush volume, can be determined based on the location of the therapeutic bolus delivery within the intrathecal space 114. The flush volume corresponds to the CSF volume between the location of the therapeutic bolus delivery and the target area. In some examples, the CSF volume can be estimated by the CNS computational or in vitro model or can be determined by imaging of the patient. Further, the total flush time can then be calculated by dividing the flush volume by the predetermined flush rate(s).

If desired, in another example, a method as described herein can include infusing a volume of flush fluid at one or more flush flow rates into the intrathecal space 114 of a patient after infusion of a therapeutic bolus, where the volume of flush fluid corresponds to the CSF volume between the location of the therapeutic bolus delivery and a target area. If desired, the method can include one or more of the examples described herein, including determination of the one or more flush flow rates based on at least one of patient anatomy or physiology data, as well as utilizing the system 100.

Turning back to the system 100 discussed above, the algorithmic infusion can be delivered through one of the example fluid delivery devices 104 using the pump device 102. For example, the first fluid port 142 of the first lumen 144 can be the therapeutic bolus delivery port and the second fluid port 144 of the second lumen 146 can be the flush fluid delivery port 148. With this configuration, one syringe 118 of the pump device 102 can contain a drug 134, such as AAV, and be fluidly connected to the first lumen 144 and the other syringe 118 of the pump device 102 can contain a flush fluid 136, such as artificial CSF, and be fluidly connected to the second lumen 146. Alternatively, in an example using a catheter 104 with a single lumen 144, the catheter 104 can be withdrawn to position the first fluid port 142 at a the second location for the flush fluid delivery, and the syringes 118 can be sequentially connected to the first lumen 144.

For one particular algorithmic infusion, the catheter 104 can be threaded within the intrathecal space 114 through a lumbar puncture access location 112 to position the therapeutic delivery fluid port 142 from a lumbar region up to a cisterna magna region, or a thoracic region up to a cervical region, and position the flush fluid port 142, 148 at a lumbar region up to a cisterna magna region, or a lumbar region up to a cervical region.

Once the controller 108 determines, receives, or retrieves the algorithmic infusion, the processor 130 carries out the infusion stored as computing logic 138 by executing the computer-readable instructions. For example, the algorithmic infusion may include instructions for the processor 130 to actuate the drivers 128 and infuse the fluid 134, 136 into the intrathecal space 114 at the therapeutic delivery and fluid delivery locations. In such an example, the instructions can include first instructions to expel the therapeutic bolus 134 from the first syringe 118. Responsive to these instructions, the driver 128 operates to drive the plunger rod 122 to move through the barrel 120 a predetermined amount according to a desired drug volume. Movement of the plunger rod 122 expels the therapeutic bolus 134 from the barrel 120 and into the intrathecal space 114 at the therapeutic delivery location of the first fluid port 142 of the catheter 104. The instructions can further include second instructions to expel the flush fluid 136 from the second syringe 118. Responsive to these instructions, the driver 128 operates to drive the plunger rod 122 to move through the barrel 120 a predetermined amount according to a desired flush fluid volume. Movement of the plunger rod 122 expels the flush fluid 136 from the barrel 120 and into the intrathecal space 114 at the flush fluid delivery location of the second fluid port 142, 148 of the catheter 104.

Moreover, as the controller 108 operates the pump device 102 to deliver the therapeutic fluid 134 and flush fluid 136 according to the algorithmic infusion, the controller 108 can also monitor CSF pressure data received from the pressure sensor 106. In some cases, the controller 108 may then compare the measured pressure to a stored, threshold pressure or range and determine if the measured CSF pressure is greater than, less than, or equal to the stored, threshold pressure. If the controller 108 determines that the measured pressure is greater than or less than a stored, threshold pressure, then the controller 108 can transmit a stop or a flush rate reduction signal to the processor 130, which causes the processor 130 to stop or control the first and/or second driver 128 to stop fluid flow from the pump device 102 or to reduce the rate of translation of the plunger rod 122 within the barrel 120 to thereby reduce the infusion flow rate.

In one example, the flush fluid 136 can be an artificial CSF or other anti-inflammatory fluid. It has been found that larger infusion bolus volumes of saline can potentially result in inflammation in the CNS tissue. Due to the large flush volumes of the methods provided herein relative to prior flush methods, utilizing an artificial CSF or other anti-inflammatory fluid can prevent or reduce an inflammation response in the patient.

In another example, a contrast agent such as imaging agent/tracer or radiocontrast agent can be added to the therapeutic bolus to allow a clinician to visualize the spread of the therapeutic bolus during and after the algorithmic infusion and/or the flush fluid to visualize the spread of the flush fluid during and after the algorithmic infusion. In particular, the contrast agent may be iohexol, iodixanol, ioversol, or barium sulfate.

In another example, a predetermined volume of air can be infused along with the therapeutic bolus and/or flush fluid, either before or after. The air pocket resulting from the infusion of air can be utilized, in combination with positioning the spine orientation and/or curvature, to block and/or limit biodistribution of the therapeutic bolus and/or flush fluid along the neuroaxis.

In another example, the patient can be positioned in a head down tilt orientation, e.g., at an angle relative to horizontal such that the patient's head is below the patient's feet. In the head down tilt orientation, the patient can be position at an angle from 0 to 90 degrees from supine position. Utilizing this orientation, a buffer can be added to the therapeutic bolus to add characteristics to the therapeutic bolus to move the bolus rostrally after infusion. In one example, the buffer can be cold relative to a patient's CSF temperature, so that the relatively colder therapeutic bolus with buffer will drop within the CNS after infusion. For example, the buffer can have a temperature in the range of 0 to 3.5 degrees C. lower than body temperature, and so forth. In another example, the buffer can cause the therapeutic bolus to have a density greater than the patient's CSF, so that the relatively more dense therapeutic bolus with buffer will drop within the CNS after infusion.

In another example, the patient can be position in a head up tilt orientation, e.g., at an angle relative to horizontal such that the patient's head is above the patient's feet. In the head up tilt orientation, the patient can be position at an angle from 1 to 90 degrees upward from supine position. It is understood that the craniospinal axis (CSA) is flexible when a person is standing up or has an otherwise head up tilt orientation, which causes the dural sac to stiffen and the cranial component to be relatively less stiff. Due to this relative flexibility and the closed fluid nature of the CNS, an infused fluid is more like to move rostrally given the potential for expansion with more flexible tissue surrounding the brain when in head-up-tilt position.

The systems and methods described herein are suitable for administering any therapeutic bolus and/or flush fluid composition, such as a pharmaceutical composition comprising one or more therapeutic agents, to a subject. Indeed, the device of the disclosure optionally comprises one or more dosages of a therapeutic agent, such as a therapeutic agent suitable for treating (in whole or in part) a disorder, infection, or injury of the central nervous system or spine. For example, the disclosure provides a method of treating Huntington's disease, Spinal Muscular Atrophy (SMA), survival motor neuron (SMN) deficiency, pain, amyotrophic lateral sclerosis (ALS) (including superoxide dismutase 1 (SOD1)-related ALS), multiple sclerosis (e.g., primary progressive multiple sclerosis), Angelman's syndrome, Dravet syndrome, Alzheimer's disease and other tau protein-related disorders, progressive supranuclear palsy (PSP), frontotemporal dementia (FTD), epilepsy, epilepsy pre-visualase, migrane, acute disseminated encephalomyelitis, acute repetitive seizures, meningitis (e.g., neoplastic meningitis), alpha-synuclei-related disorders including Parkinson's Disease, cancer (e.g., central nervous system lymphoma, leptomeningeal cancer, or secondary malignant neoplasms (SMN)), inflammation, Sanfilippo A or B, Friedreich's Ataxia, hereditary cerebral hemorrhage with amyloidosis-Dutch type (HCHWA-D), cerebral amyloid angiopathy (CAA), Rett Syndrome and other x-linked CNS disorders, Duchenne Muscular Dystrophy, Friedreich's Ataxia, Spinal Muscular Atrophy with respiratory distress (SMARD1), CLN8, or amyloid congophilic angiopathy (ACA) using the system described herein to deliver a therapeutic to a desired anatomical site (e.g., the intrathecal space).

The plurality of syringes 118 may house a variety of fluids within the barrel 120. For example, the fluid may comprise a therapeutic agent such as a nucleic acid, a protein therapeutic, a cell therapy, a small molecule, a viral vector encoding a therapeutic protein, or a combination thereof.

Examples of protein therapeutics include antibody-based therapeutics, such as antibodies, antibody fragments, or antibody-like protein products that include binding regions of antibodies (e.g., scFv, diabodies, antibody mimetics, and the like). The antibody-based therapeutic may target, e.g., amyloid plaques, tau proteins, cancer antigens, or abnormal alpha-synuclein. Examples of protein therapeutics also include, but are not limited to, hormones, enzymes (e.g., lysosomal enzymes, such as alpha-L-iduronidase, N-acetylgalactosamine-4-sulfatase, or beta-glucuronidase), growth factors (e.g., fibroblast growth factor (FGF) or neurotrophins or neurotrophic factors, such as glial cell-derived neurotrophic factor (GDNF), brain-derived neurotrophic factor (BDNF), ciliary neurotrophic factor (CNTF), or nerve growth factor (NGF)), blood factors, bone morphogenetic proteins, interferons, interleukins, and thrombolytics. Examples of cell-based therapies include, but are not limited to, stem cell therapeutics and immune cells (including modified immune cells, such as CAR T cells). Suitable small molecule therapeutics include, but are not limited to, analgesics, ion channel blockers, anti-convulsive agents, antibiotics or antiviral agents, anti-inflammatories, anticoagulants, chemotherapeutic, anti-depressants, anti-anxiety agents, steroids, and the like. In various aspects, the therapeutic agent is baclofen, morphine, bupivacaine hydrochloride, clonidine hydrochloride, gabapentin, idursulfase, cytarabine, methotrexate, a corticosteroid, edavarone-conjugate, conotoxin, abomorphine, prednisolone hemisuccinate sodium, carbidopa/levodopa, tetrabenazine, benzodiazepines, such as diazepam and midazolam, alphaxalone or other derivative, cyclophosphamide, idursulfase (Elaprase®), iduronidase (Aldurazyme®), topotecan, buslfan, opmaveloxolone, epicatechin, methylprednisolone, frataxin replacement, reservatrol, nicontinamide, AT-010 (RNA that induces splicing modulation in the mature amyloid precursor protein mRNA), Cerebril™, an anti-A□ antibody, elenbecestat, a corticosteroid, or nusinersen (Spinraza®), or combinations thereof.

Nucleic acid therapeutics include DNA or RNA, which may be single stranded or double stranded and which may be modified or unmodified. In particular, the nucleic acid may be an antisense oligonucleotide, a ribozyme, an miRNA, an siRNA, and shRNA, or a nucleic acid encoding a clustered regularly interspaced short palindromic repeats (“CRISPR”) associated protein (Cas) system, or combination thereof. The CRISPR/Cas system is further described in, e.g., U.S. Patent Publication Nos. 2018/0223311.

Optionally, the nucleic acid targets a gene (or gene product, such as mRNA) selected from the group consisting of APP, MAPT, SOD1, BACE1, CASP3, TGM2, TARDBP, ADRB1, CAMK2A, CBLN1, CDK5R1, GABRA1, MAPK10, NOS1, NPTX2, NRGN, NTS, PDCD2, PDE4D, PENK, SYT1, TTR, FUS, LRDD, CYBA, ATF3, CASP2, HRK, C1QBP, BNIP3, MAPK8, MAPK14, Rac1, GSK3B, P2RX7, TRPM2, PARG, CD38, STEAP4, BMP2, GJA1, TYROBP, CTGF, ANXA2, DUOX1, RTP801, RTP801L, NOX4, NOX1, NOX2 (gp91pho, CYBB), NOX5, DUOX2, NOXO1, NOXO2 (p47phox, NCF1), NOXA1, NOXA2 (p67phox, NCF2), p53 (TP53), HTRA2, KEAP1, SHC1, ZNHIT1, LGALS3, SESN2, SOX9, ASPP1, CTSD, CAPNS1, FAS, FASLG, CAPN1, FADD, CASP1, CASP9, p75NTR, PARK2, HTT (with expanded repeats), NogoA, MAG, OMGP, NgR1, PDE4, BCAN, NCAN, PTPRZ1, TNC, NRP1, NRP2, PLXNA1, PLXNA2, PLXNB1, PLXNC1, TROY, LRRC1, ROCK1, LimK1, LimK2, CFL1, KCNC4, KCNE3, NAT8L, FKBP1A, FKBP4, LRRK2, DYRK1A, AKAP13, UBE2K, WDR33, MYCBP2, SEPHS1, HMGB1, HMGB2, TRPM7, BECN1, THEM4, SLC4A7, MMP9, SLC11A2, ATXN3, ATXN1, ATXN7, PRNP, EFNB3, EPHA4, EFNAS, EPHA7 and EFNB2, such that gene expression or function is modified.

In some embodiments, the therapeutic agent is an oligonucleotide comprising at least one modified nucleotide, optionally a modified nucleotide that reduces binding to cerebral spinal fluid (CSF) proteins. In various embodiments, the modified nucleotide includes a substituent at the 2′-position, such as a 2′-O-2-methoxyethyl (“2′-MOE”) group, as shown below, wherein X is 0 or S.

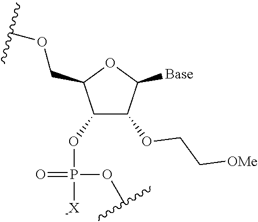

Oligonucleotides comprising a 2′-MOE modification can distribute rapidly in central nervous system tissues. Oligonucleotides comprising such modifications exhibit extended half-lives in CSF and central nervous system tissues, which can result in less frequent dose administration.

In some cases, the modified nucleotide can include a 2′,4′-constrained group, such as a constrained 2′-O-ethyl (“cEt”) group. In various cases, the cEt group can have S-stereochemistry (“S-cEt”), as shown below, wherein X is O or S.

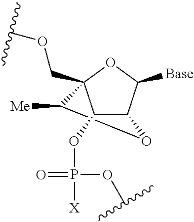

Nucleic acids modified with a constrained ethyl group, such as S-cEt, can exhibit enhanced thermal stability, good potency, and a good therapeutic profile.

In various embodiments, the nucleic acid is an antisense nucleic acid reduces expression of HTT (e.g., HTT with expanded repeats). The sequence of HTT is known, see, e.g., GenBank Accession No. NM_002111. In some embodiments, the nucleic acid is an antisense nucleic acid that targets mRNA encoding Huntington protein (HTT), such as mutant HTT (e.g., HTT with expanded repeats). In various aspects, the nucleic acid comprises the nucleic acid sequence ctcagtaacattgacaccac. In various aspects, the nucleic acid is used in connection with the device in a method of treating Huntington's disease.

In various aspects, the nucleic acid is a modified antisense oligonucleotide that targets survival motor neuron-2 (SMN2) mRNA, optionally the intron downstream of exon 7 of the SMN2 transcript. The sequence of SMN2 is known, see, e.g., GenBank Accession No. NM_022876. Optionally, the antisense oligonucleotide is modified such that the 2′-hydroxy groups of the ribofuranosyl rings are replaced with 2′-O-2-methoxyethyl groups and the phosphate linkages are replaced with phosphorothioate linkages. In various aspects, the nucleic acid is nusinersen and is used in connection with the device in a method of treating spinal muscular atrophy (SMA).

Optionally, the nucleic acid encodes a beneficial protein that, e.g., replaces an absent or defective protein, or encodes a cytotoxic protein that achieves a therapeutic effect, such as cancer cell death. Any of the protein-based therapeutics described herein may be delivered to a subject via delivery of a nucleic acid encoding the protein under conditions which allow expression in vivo. For example, in various embodiments, the nucleic acid encodes a neurotrophic factor such as, but not limited to, nerve growth factor (NGF), brain-derived neurotrophic factor (BDNF), neurotrophin-3 (NT-3), neurotrophin-4/5 (NT-4/5), neurotrophin-6 (NT-6), ciliary neurotrophic factor (CNTF), glial cell line-derived neurotrophic factor (GDNF), the fibroblast growth factor family (e.g., FGF's 1-15), leukemia inhibitory factor (LIF), certain members of the insulin-like growth factor family (e.g., IGF-1), a neurturin, persephin, a bone morphogenic protein (BMPs), an immunophilin, a member of the transforming growth factor (TGF) family of growth factors, a neuregulin, epidermal growth factor (EGF), platelet-derived growth factor (PDGF), vascular endothelial growth factor family (e.g. VEGF 165), follistatin, or Hifl, or combinations thereof.

Optionally, the fluid comprises a gene expression (e.g., viral) vector. Examples of viral vectors include, e.g., herpes simplex virus (HSV) vectors, adenovirus (Ad) vectors, parvoviral-based vectors (e.g., adeno-associated viral vectors), chimeric Ad-AAV vectors, and retroviral vectors (including lentiviral vectors, HIV vectors). In some embodiments, the viral vector is an AAV vector and all serotypes. In some examples, the therapeutic agent can include AAV vector DNA sequences, recombinant AAV particles, or combinations thereof. In further examples, the AAV vector DNA sequences can target mRNA encoding a MECP2 gene and/or AAV containing miRNA sponge sequences that interfere with miRNA 106a and Xist on the silent X-chromosome thereby slightly unfolding the chromosome and allowing re-expression of silent healthy gene. In some examples, the gene expression vector can be infused according to a therapy platform, where the therapy platform can be one of convention gene replacement, x-reactivation, exon skipping, or promoter modulation.

Alternatively or in addition, the fluid disposed within the barrel 120 may be a diagnostic agent. The fluid may be a contrast media such as imaging agent or radiocontrast agent. In particular, the contrast media may be gadolinium, iohexol, iodixanol, ioversol, or barium sulfate. This contrast may be added into the therapeutic bolus and/or flush fluid for real-time visualization and feedback control of the flush rate and volume to achieve a desired distribution within the CSF.

### EXAMPLES

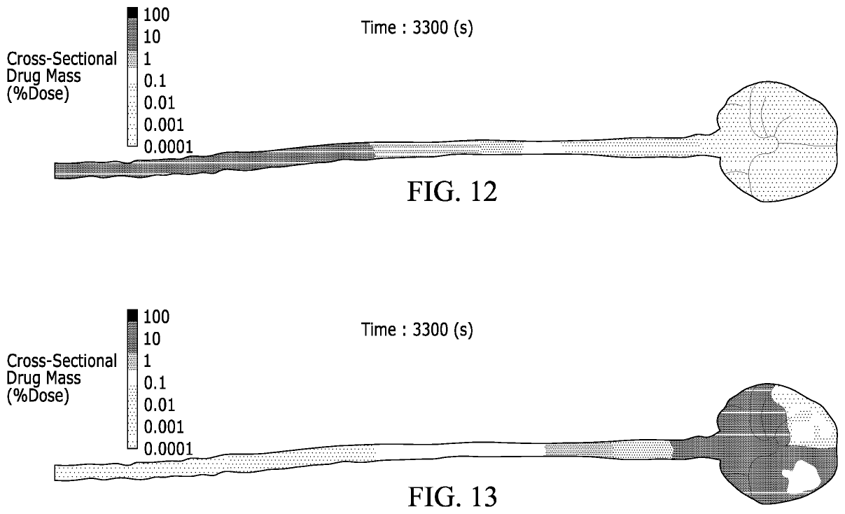

FIGS. 12 and 13 show drug mass distribution plots for example therapeutic bolus infusions to show a comparison between a lumbar puncture procedure and a procedure utilizing an algorithmic infusion according to a disclosure herein.

In the lumbar puncture control, shown in FIG. 12, a simulated therapeutic bolus was infused using a lumbar puncture procedure in the lumbar region of the spine. The drug mass distribution plot for this procedure shows distribution of the therapeutic bolus after 3300 seconds. As explained by the key, the brain shows a cross-sectional drug mass of around 0.0001, the cervical region has a range of cross-sectional drug mass between 0.0001 to 0.01, the thoracic region has a range of cross-sectional drug mass between 0.01 to above 1, and the lumbar region has a cross-sectional drug mass of 1 and above.

In the algorithmic infusion example, shown in FIG. 13, a simulated therapeutic bolus was infused in the thoracic region of the spine and the flush fluid was infused in the lumbar region of the spine. The drug mass distribution plot for this procedure also shows distribution of the therapeutic bolus after 3300 seconds. As explained by the key, portions of the brain and the cervical region of the spine include a cross-sectional drug mass of above 0.1 and many areas of 1 and above. Further, the thoracic region has a range of cross-sectional drug mass between 1 and below 0.01, while the lumbar region has a cross-sectional drug mass below 0.01 and caudal areas below 0.001.

Accordingly, comparing FIGS. 12 and 13, utilizing an algorithmic infusion as described herein vastly and unexpectedly increases cross-sectional drug mass in the target area, e.g., the brain as shown, or other desired target areas, by upwards of 1,000,000 percent. These drastic results show that a therapeutic bolus can be accurately targeted to a specific area in the central nervous system. Advantageously, this targeted approach can decrease bolus size and/or reduce a number of infusions, among other benefits.

It will be appreciated that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. The same reference numbers may be used to describe like or similar parts. Further, while several examples have been disclosed herein, any features from any examples may be combined with or replaced by other features from other examples. Moreover, while several examples have been disclosed herein, changes may be made to the disclosed examples within departing from the scope of the claims.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

1. A method for fluid delivery to a target area in a central nervous system of a patient, the method comprising:
infusing a therapeutic bolus into an intrathecal space of a patient in a first location;
subsequently infusing a flush fluid into the intrathecal space of the patient in a second location at one or more flush flow rates, the one or more flush flow rates based on at least one of patient anatomy or physiology data.

2. The method of claim 1, wherein the one or more flush flow rates are calculated using:
estimated or measured steady-streaming fluid velocities of cerebrospinal fluid within the intrathecal space of the patient between the first location and the target area divided into one or more axial sections; and
estimated or measured axial cross-sectional areas of the one or more axial sections.

3. The method of claim 2, wherein the one or more flush flow rates are based on averages of the steady-streaming fluid velocities or maximum values of the steady-streaming fluid velocities in the one or more axial sections.

4. The method of claim 3, wherein the one or more flush flow rates are calculated using a predetermined percentage of the averages of the steady-streaming fluid velocities or the maximum values of the steady-streaming fluid velocities in the one or more axial sections.

5. The method of claim 1, wherein the at least one of patient anatomy or physiology data comprises data obtained from patient imaging and tests, and computations performed on the patient imaging and tests.

6. The method of claim 1, wherein the at least one of patient anatomy or physiology data comprises one or more of: patient age, patient sex, patient size, patient CSF volume, patient CSF dynamics, patient respiration data, patient sleep data, patient anatomical geography, heart rate, or disease.

7. The method of claim 6, wherein the one or more steady-streaming fluid velocities are estimated by a central nervous system computational or in vitro model for the patient using the at least one of patient anatomy or physiology data as input.

8. The method of claim 1, wherein a volume of the flush fluid corresponds to a volume of the cerebrospinal fluid between the first location and the target area.

9. The method of claim 8, further comprising imaging the patient to determine the volume of the cerebrospinal fluid between the first location and the target area.

10. The method of claim 1, wherein the target area is the brain, the spine or combinations thereof.

11. The method of claim 1, further comprising:
measuring CSF pressure of the patient with a pressure sensor; and
stopping the infusion of the flush fluid or reducing the one or more flush flow rates in response to determining that the CSF pressure exceeds a predetermined threshold or drops below a predetermined threshold.

12. The method of claim 1, further comprising:
measuring CSF pressure of the patient with a pressure sensor; and
beginning the infusion of the flush fluid in response to determining that the CSF pressure indicates an ascending or descending phase of a waveform.

13. The method of claim 1, wherein the second location is spaced caudally from the first location.

14. The method of claim 1, wherein the first location and the second location are in a same region of the intrathecal space.

15. The method of claim 1, further comprising:
creating an access opening into the intrathecal space of the patient in a lumbar region thereof; and
inserting a catheter through the access opening and threading the catheter rostrally within the intrathecal space, wherein infusion of the therapeutic bolus and the flush fluid is performed through the catheter.

16. The method of claim 15, wherein the catheter comprises a first lumen with a first fluid port and a second lumen with a second fluid port, the second fluid port spaced proximally of the first fluid port, and threading the catheter rostrally within the intrathecal space positions the first fluid port at the first location within a lumbar region up to a cisterna magna region for infusion of the therapeutic bolus and positions the second fluid port at the second location within the lumbar region up to the cisterna magna for infusion of the flush fluid.

17. The method of claim 16, wherein threading the catheter rostrally within the intrathecal space positions the first fluid port at the first location within a thoracic region up to the cervical region of the intrathecal space for infusion of the therapeutic bolus and positions the second fluid port at the second location within the lumbar region up to the cervical region of the intrathecal space for infusion of the flush fluid.

18. The method of claim 16, wherein threading the catheter rostrally within the intrathecal spaced comprises moving the first lumen relative to the second lumen within the intrathecal space.

19. The method of claim 15, wherein the catheter comprises a single lumen with a distal fluid port, and threading the catheter rostrally within the intrathecal space comprises:
positioning the distal fluid port at the first location within a lumbar region up to a cisterna magna region for infusion of the therapeutic bolus; and
withdrawing the catheter to position the distal fluid port at the second location within the lumbar region up to the cisterna magna for infusion of the flush fluid.

20. The method of claim 19, wherein threading the catheter rostrally within the intrathecal space comprises:
positioning the distal fluid port at the first location within a thoracic region up to a cervical region for infusion of the therapeutic bolus; and
withdrawing the catheter to position the distal fluid port at the second location within the lumbar region for infusion of the flush fluid.

21. The method of claim 1, further comprising:
creating an access opening into the intrathecal space of the patient in a lumbar region thereof with an introducer needle; and
inserting a catheter through the access opening and threading the catheter rostrally within the intrathecal space, wherein infusion of the therapeutic bolus is performed through the introducer needle and the infusion of the flush fluid is performed through the catheter.

22. The method of claim 1, further comprising creating an access opening into the intrathecal space of the patient in a lumbar region thereof with a lumbar puncture needle, wherein infusion of the therapeutic bolus and the flush fluid is performed through the lumbar puncture needle in the lumbar region.

23. The method of claim 1, further comprising piercing a septum of a plug-in port implanted in the patient with a needle, and wherein infusion of at least one of the therapeutic bolus or the flush fluid is performed through the plug-in port.

24. The method of claim 1, wherein infusing the flush fluid comprises infusing an artificial cerebrospinal fluid or anti-inflammatory fluid into the intrathecal space of a patient in the second location at the one or more flush flow rates.

25. The method of claim 1, further comprising positioning the patient in a head up tilt orientation prior to infusing the therapeutic bolus and infusing the flush fluid.

26. The method of claim 1, further comprising:
positioning the patient in a head down tilt orientation prior to infusing the therapeutic bolus and infusing the flush fluid; and
wherein the therapeutic bolus comprises a buffer, the buffer being at least one of: colder than a temperature of cerebrospinal fluid of the patient; or more dense than the cerebrospinal fluid of the patient.

27. The method of claim 1, wherein at least one of the therapeutic bolus or the flush fluid comprises a contrast agent; and further comprising adjusting the one or more flush flow rates based on real-time feedback from the contrast agent.

28. The method of claim 1, wherein the therapeutic bolus comprises a nucleic acid, a protein therapeutic, a cell therapy, a small molecule therapeutic, a viral vector encoding a therapeutic protein, or a combination thereof.

29. The method of claim 28, wherein therapeutic bolus comprises the nucleic acid selected from the group consisting of an antisense oligonucleotide, a ribozyme, an miRNA, an siRNA, and shRNA, or a nucleic acid encoding a clustered regularly interspaced short palindromic repeats (CRISPR) associated protein (Cas) system, or a combination thereof.

30. The method of claim 28, wherein the therapeutic bolus comprises an antisense oligonucleotide that targets mRNA encoding Huntington protein (HTT) or an antisense oligonucleotide that targets mRNA encoding survival motor neuron-2 (SMN2).

31. The method of claim 1, wherein the therapeutic bolus comprises adeno-associated virus (AAV) vector DNA sequences, recombinant AAV particles, or combinations thereof.

32. The method of claim 31, wherein the AAV vector RNA sequences encode miRNA sponge sequences that interfere with miRNA 106a and Xist on the silent X-chromosome thereby slightly unfolding the chromosome and allowing re-expression of the silent healthy gene.

33. The method of claim 1, wherein the therapeutic bolus is infused according to a therapy platform comprising one of: conventional gene replacement, x-reactivation, exon skipping, or promoter modulation.

34.-102. (canceled)
